(19) United States
(12) Patent Application Publication
(10) Pub. No.: US 2018/0264049 A1
(43) Pub. Date: Sep. 20, 2018

(54) Immunotherapy of Aberrant Ocular Angiogenesis by Placental Vaccination

(71) Applicant: Batu Biologics, Inc., San Diego, CA (US)

(72) Inventors: Samuel C. Wagner, San Diego, CA (US); Thomas E. Ichim, San Diego, CA (US)

(21) Appl. No.: 15/924,595

(22) Filed: Mar. 19, 2018

Related U.S. Application Data
(60) Provisional application No. 62/473,927, filed on Mar. 20, 2017.

(51) Int. Cl. A61K 35/50; A61P 27/02

(52) U.S. Cl. CPC A61K 35/50; A61P 27/02

(57) ABSTRACT

Disclosed are materials, methods, and protocols for inducing a therapeutic response in ocular conditions, such as diabetic retinopathy and wet macular degeneration, by immunizing with placental endothelial cells. In one embodiment, the invention teaches the administration of placental endothelial cells or products thereof, in an immunogenic context to induce antibody and/or cellular immune responses towards ocular neovascularization. The immunogenicity may be endowed by treatment with interferon gamma or agents known to induce upregulation of HLA. In one embodiment, the invention produces antibodies selectively targeting VEGF-associated angiogenesis characterized by a higher degree of vascular permeability as compared to non-malignant angiogenesis. In another embodiment, the invention provides means of inducing antibodies to vasculature associated with aberrant ocular angiogenesis through immunization with endothelial cells grown in conditions resembling tumor endothelial cells.

### CROSS-REFERENCE TO RELATED APPLICATIONS

This Patent Application takes priority from Provisional Patent Application No. 62/473,927, titled Immunotherapy of Aberrant Ocular Angiogenesis by Placental Vaccination, filed on Mar. 20, 2017, the contents of which are expressly incorporated herein by this reference as though set forth in their entirety and to which priority is claimed.

### FIELD OF THE INVENTION

The invention pertains to the field of ocular disorders. More specifically, the invention provides means of treating and/or preventing diabetic retinopathy or wet macular degeneration.

### BACKGROUND OF THE INVENTION

Macular degeneration is the general term for a disorder in which a part of the retina called the macula deteriorates. Age-related macular degeneration (AMD) is the most common type of macular degeneration. It has been reported that in the United States, AMD is the leading cause of blindness in people older than 55. More than 10 million people in the US are affected by this disease, which includes 23% of people over 90, as noted by WebMD. (www.webmd.com/eye-health/macular-degeneration/macular-degeneration-overview).

There are various types of macular degeneration that afflict patients. One type of macular degeneration is “dry” macular degeneration, also called geographical atrophy. Dry macular degeneration is an early stage of the disorder in which drusen is deposited on the macula at a subretinal level. The deposition of this drusen may result from aging or thinning of the macular tissues. As a result of this deposition of drusen, loss of central vision may gradually occur. Many times, AMD begins with dry macular degeneration.

Another type of AMD is “wet” macular degeneration. Wet macular degeneration is a neovascular type of degeneration in which incomplete blood vessels abnormally grow under the retina and begin to leak. As a result of this leakage, permanent damage occurs to photoreceptor cells of the retina which ultimately causes the death of these cells and thus, blind spots. Unlike dry macular degeneration, in which the vision loss may be minor, the vision loss that occurs in wet macular degeneration can be severe. Indeed, it has been reported that although only 10% of those with AMD suffer from wet macular degeneration, 66% of those with AMD suffering from significant visual loss can directly attribute that loss to wet macular degeneration.

Retinopathies such as wet macular degeneration and diabetic retinopathies cause regions of leakage induced edema within the retina. To compensate for the lack of oxygen and nutrients in the ischemic tissue, cytokines involved in permeability are excessively up-regulated. The over-expression of vascular endothelial growth factor (VEGF) causes an increase in vessel permeability due to depletion of cell-to-cell adhesion molecules, such as VE-cadherin and claudin-5. This leads to aberrant angiogenesis and hyperproliferative activities of the neovasculature, leading to damage to the retinal nerve and eventual blindness. The increased permeability of vasculature in the eye can result in edema. While clinical characterization of the causes of edema such as diabetes have been studied, little attention has been paid to addressing the weakened inter-cellular junctions in retinal vasculature. The edema associated with vasculature leakage can cause complications such as macular edema and exudative retinal detachment.

Currently, there is a good progress in the treatment of wet macular degeneration. Treatment with VEGF antagonists largely replaced an earlier method of treating this condition by laser-cauterizing hemostasis. However, due to the poor effect of VEGF antagonists treatment, photodynamic therapy has also been used. Although photodynamic therapy has an improved efficacy, it is still unsatisfactory. Recently, a new VEGF antagonist—Lucentis, which is a recombinant of human-derived VEGF subtype monoclonal antibody fragment, has beend developed and it could reduce angiogenesis. This medicament was approved by U.S. FDA for treating wet macular degeneration in 2006, and has a good efficacy. Meanwhile, it was found that this anti-VEGF drug also has therapeutic effect on diabetic retinopathy and neovascular glaucoma. However, since Lucentis is an antibody drug with an extremely high price, it is not readily available all over the world. Therefore, there is intense competitive focus in the current international pharmaceutical industry to develop novel means of inhibiting ocular angiogenesis having excellent efficacy and low price.

Diabetic retinopathy (DR) is recognized as a retinal vascular disorder that includes: (1) excess capillary permeability, (2) vascular closure, and (3) proliferation of new vessels. DR is recognized to consist of two (2) stages: nonproliferative and proliferative. In the nonproliferative stage, the disease is characterized by a loss of retinal capillary pericytes, thickening of the basement membrane and development of microaneurysms, dot-blot hemorrhages, and hard exudates. In the proliferative stage, patients develop extensive neovascularization, vessel intrusion into the vitreous, bleeding and fibrosis with subsequent retinal traction, which leads to severe vision impairment. While the pathological stages of diabetic retinopathy are well-described, the molecular events underlying diabetic retinopathy are poorly understood. This is due, in part, to the fact that the disease progresses over ten to thirty years, depending on a given individual. Tight control of glycemia and hypertension and ophthalmic screening of diabetics appears beneficial in preventing the disease. Current treatment consists of regular observation by an ophthalmologist, laser photocoagulation and vitrectomy. Unfortunately, these approaches are limited by various adverse effects and incomplete efficacy.

### DESCRIPTION OF THE PREFERRED EMBODIMENTS

The invention teaches generation of cellular and humoral responses towards antigens found on aberrant angiogenesis cells in an ocular environment. It is known that in conditions such as wet macular degeneration, an excess of proliferating cells occur in the endothelial lineage. While treatment with VEGF inhibitors and antibodies has been somewhat successful, adverse effects include high ocular pressure and retinal nerve damage. The invention overcomes these by providing a stable immunogen that does not require continued administration, but instead focuses the immune system to continually produce antibodies, as well as cellular responses to suppress aberrant angiogenesis but not physiological angiogenesis. This is based on similarities between proliferative endothelial cells and cells in the ocular pathological environment. One common factor is the dependence on VEGF, which creates blood vessels that are predominantly permeable.

In one specific embodiment of the invention, endothelial cells are derived from placental tissue, isolated into a homogeneous or semi-homogeneous mixture, treated with agents capable of augmenting immunogenicity, and subsequently administered into a recipient in which immune response to proliferating endothelium is desired. In one specific example, endothelial cells are purified from a human placenta according to the following steps:

a) Fetal membranes are manually peeled back and the villous tissue is isolated from the placental structure, with caution being used not to extract the deciduas or fibrous elements of the placental structure;

b) The fetal villous tissue is subsequently washed with cold saline to remove blood and scissors are used to mechanically digest the tissue into pieces as small as possible;

c) The minced tissue is then enzymatically digested. Specifically, about 25 grams of minced tissue is incubated with approximately 56 ml of liquid solution which has been pre-warmed to a temperature of 37 Celsius. The solution is comprised of Hanks Buffered Saline Solution (HBSS) supplemented with 25 mM of HEPES and containing Calcium and Magnesium, the solution containing 0.28% collagenase, 0.25% dispase, and 0.01% DNAse (added during the incubation periods as described below);

d) The mixture of minced placental villus tissue and digesting solution is incubated under stirring conditions for three incubation periods of 20 minutes each. Ten minutes after the first incubation period and immediately after the second and third incubation periods, the DNAse is added to make up a total concentration of DNase, by volume, of 0.01%;

e) In the first and second incubations, the incubation flask is set at an angle, and the tissue fragments are allowed to settle for approximately 1 minute, with 35 ml of the supernantant cell suspension being collected and replaced by 38 ml (after the first digestion) or 28 ml (after the second digestion) of fresh digestion solution. After the third digestion the whole supernatant is collected;

f) The supernatant collected from all three incubations is pooled and is poured through approximately four layers of sterile gauze and through one layer of 70 micro meter polyester mesh. The filtered solution is then centrifuged for 1000 g for 10 minutes through diluted new born calf serum, the new born calf serum diluted at a ratio of 1 volume saline to 7 volumes of new born calf serum;

g) The pooled pellet is then resuspended in 35 ml of warm DMEM with 25 mM HEPES containing 5 mg DNase I;

h) The suspension is then mixed with 10 ml of 90% Percoll to give a final density of 1.027 g/ml and is centrifuged at 550 g for 10 minutes with the centrifuge brake off;

i) The pellet is then collected and resuspended in 15 ml of DMEM with 25 mM HEPES that is layered over a discontinuous Percoll gradient comprising of 20%-70% Percoll in 10% steps and centrifuged at 1900 g for 20 minutes;

j) The cells found at the 1.037 g/ml and 1.048 g/ml are collected utilized for the generation of a cellular vaccine product.

The cellular vaccine product from step “j”, in a preferred embodiment is treated with an agent capable of augmenting immunogenicity. The immunogenicity in this context refers to ability to enhance recognition by recipient immune system. In one embodiment, immunogenicity refers to enhanced expression of HLA I and/or HLA II molecules. In another embodiment, immunogenicity refers to enhanced expression of costimulatory molecules. The costimulatory molecules are selected from a group comprising: CD27; CD80; CD86; ICOS; OX-4; and 4-1 BB. In another embodiment, immunogenicity refers to enhanced ability to stimulate proliferation of allogeneic lymphocytes in a mixed lymphocyte reaction. Immunogenicity may be augmented by incubation with one of the lymphokine or cytokine proteins that are known in the art, or with a member of the interferon family.

In one particular embodiment, the purified endothelial cells are incubated with interferon gamma. In one particular embodiment, interferon gamma is incubated with endothelial cells, whether purified or unpurified for a period of approximately 48 hours, at a concentration of approximately 150 IU/ml. Endothelial cells may be expanded after purification as described above before treatment with agents capable of augmenting immunogenicity. For example, endothelial cells may be treated with an endothelial cell mitogen. The endothelial cell mitogen may be any protein, polypeptide, variant or portion thereof that is capable of, directly or indirectly, inducing endothelial cell growth.

Such proteins include, for example, acidic and basic fibroblast growth factors (aFGF) (GenBank Accession No. NP-149127) and bFGF (GenBank Accession No. AAA52448), vascular endothelial growth factor (VEGF) (GenBank Accession No. AAA35789 or NP-001020539), epidermal growth factor (EGF) (GenBank Accession No. NP-001954), transforming growth factor α (TGF-α (GenBank Accession No. NP-003227) and transforming growth factor β (TFG-β) (GenBank Accession No. 1109243A), platelet-derived endothelial cell growth factor (PD-ECGF) (GenBank Accession No. NP-001944), platelet-derived growth factor (PDGF) (GenBank Accession No. 1109245A), tumor necrosis factor α (TNF-α) (GenBank Accession No. CAA26669), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP-000749), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994).

Variants or fragments of a mitogen may be used as long as they induce or promote endothelial cell or endothelial progenitor cell growth. Preferably, the endothelial cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993). Before expansion, endothelial cells may be further purified based on expression of surface receptors using affinity-based methodologies that are known to one of skill in the art, the methodologies include magnetic activated cell sorting (MACS), cell panning, or affinity chromatography. Other methodologies such as fluorescent activated cell sorting (FACS) may also be used. Various lectins are known to have selectivity to endothelial cells. For example, Ulex europaeus agglutinin I is known to possess ability to bind to endothelial cells and endothelial progenitor cells. It is within the scope of the current invention to define “endothelial cell” as including “endothelial progenitor cell”.

The vaccine formulation may be utilized in conjunction with known adjuvants in order to induce an immune response that is Th1 or Th17-like, and which will inhibit the proliferation of endothelial cells in the recipient. Such adjuvant compounds are known in the art to boost the activity of the immune system and are now under study as possible adjuvants, particularly for vaccine therapies. Some of the most commonly studied adjuvants are listed below, but many more are under development.

For example, Levamisole, a drug originally used against parasitic infections, induces cytotoxic immunity such as in CD8 cells, which is useful for destruction of neovasculature which is pathological. It is often used as an immunotherapy adjuvant because it can generally activate T lymphocytes. Additionally, the compound has been demonstrated to induce maturation of dendritic cells, further supporting an immune modulatory role. Additionally, it has been shown to augment efficacy of other immunotherapeutic agents such as interferon.

Aluminum hydroxide (alum) is one of the most common adjuvants used in clinical trials for. It is already used in vaccines against several infectious agents, including the hepatitis B virus.

In another embodiment, incomplete Freund's Adjuvant (IFA) is given together with some experimental therapies to help stimulate the immune system and to increase the immune response to cancer vaccines, both protein and peptide in part by providing a localization factor for T cells. IFA is a liquid consisting of an emulsifier in white mineral oil.

Another vaccine adjuvant useful for the present invention is interferon alpha, which has been demonstrated to augment NK cell activity, as well as to promote T cell activation and survival.

QS-21 is a relatively new immune stimulant made from a plant extract that increases the immune response to vaccines used against melanoma.

DETOX is another relatively new adjuvant. It is made from parts of the cell walls of bacteria and a kind of fat. It is used with various immunotherapies to stimulate the immune system.

Keyhole limpet hemocyanin (KLH) is another adjuvant used to boost the effectiveness of cancer vaccine therapies. It is extracted from a type of sea mollusc. Dinitrophenyl (DNP) is a hapten/small molecule that can attach to tumor antigens and cause an enhanced immune response.

In one embodiment of the invention, proliferating endothelial cells treated with an agent to stimulate immunogenicity are lysed and protein extracts are extracted and utilized as a vaccine. In some embodiments, specific immunogenic peptides may be isolated for the cell lysate. In other embodiments, lyophilization of endothelial cells is performed subsequent to treatment with an agent that augments immunogenicity.

In embodiments utilizing cellular extracts, various formulations may be generated. The formulations may conveniently be presented in unit dosage form and may be prepared by any of the methods well known in the art of pharmacy. Such methods include the step of bringing into association the active ingredient (for an antigenic molecule, construct or chimaeric polypeptide of the invention) with the carrier which constitutes one or more accessory ingredients. In general, the formulations are prepared by uniformly and intimately bringing into association the active ingredient with liquid carriers or finely divided solid carriers or both, and then, if necessary, shaping the product.

In one embodiment of the invention, ValloVax (Ichim et al, J Transl Med 2015) is used to stimulate immunity towards ocular neovascularization.

In one embodiment, endothelial cell vaccination is administered with an adjunct therapeutic: such as an anti-VEGF agent. An anti-VEGF agent herein illustratively includes bevacizumab ranibizumab small molecules that inhibit the tyrosine kinases stimulated by VEGF, such as lapatinib, sunitinib, sorafenib, axitinib, pazopanib, or a combination thereof. A combination therapeutic is provided that includes an anti-VEGF agent. It has been surprisingly found that by simultaneously suppressing VEGF binding to a cell and stimulation of Tight Junction and Adherens Junction protein expression, that the efficacy of conventional anti-VEGF agents is enhanced. By way of example, anti-VEGF agents are typically effective in approximately 75% of subject with the indication of macular edema secondary to diabetes.

In one embodiment of the invention, a condition associated with ocular endothelial hyperproliferation is treated with an anti-angiogenic vaccine combined with anti-angiogenic treatments. These treatments are a point of growing interest as the process of new blood vessel formation is known to be main factor for spread of cancers with excessive growth of blood vessels. Also, abnormal blood vessels develop under macula and break, bleed, and leak fluid, causing macular degeneration in older people. Anti-angiogenic agents have been widely used for inhibiting growth of blood vessels. They can be primarily classified into three: monoclonal antibodies, small molecule tyrosine kinase inhibitors and inhibitors of mTOR (mammalian target of rapamycin).

Vascular endothelial growth factor (VEGF) which is one of the major growth factors that control angiogenesis has become target of research community in production of anti-angiogenic drugs. Bevacizumab (Lucentis™) is a humanised monoclonal antibody that inhibits VEGF-A. It is primarily used in large doses for treating metastatic colorectal cancer, lung, breast and kidney cancers. Pegaptanib sodium (Macugen™) is another anti-angiogenic agent which is a pegylated anti-VEGF aptamer, a single strand of nucleic acid. It binds specifically to VEGF 165, a protein that plays critical role in angiogenesis. Pegatanib is developed for treating neovascular age-related macular degeneration (AMD) (Ng E W and Adamis A P, 2005). Other forms of treatments targeting VEGF have also been developed. One of the relevant prior arts in this field of technology is disclosed in International Publication no. WO2009/149205. This reference discloses a cell therapy for delivering soluble VEGF receptor to eye for treating ophthalmic and cell proliferation disorders. In this reference, new cells lines that express VEGF receptor have been developed by recombinant technology.

In one embodiment of the invention, immunization is performed in order to induce antibodies to VEGF which resemble soluble VEGF receptors. The anti-angiogenic proteins generated in the present invention produce VEGF blocking activity, which resembles soluble versions of the three types of VEGF receptors: VEGFR-1, VEGFR-2 and VEGFR-3. Vascular endothelial growth factor receptor-1 (VEGFR-1), also known as fms-related tyrosine kinase 1 (FLT-1) in human, is a receptor tyrosine kinase (RTK) specific for the angiogenic factors VEGF such as VEGF-A, VEGF-B and placental growth factor (PIGF). VEGFR-1 is expressed in two forms via alternate splicing at the pre-mRNA level: a full-length, membrane bound receptor capable of transducing signal and a truncated, soluble receptor (sVEGFR-1) capable of sequestering ligand or dimerizing with full-length receptor and preventing signal transduction. Human VEGFR-1 gene produces two major transcripts of 3.0 and 2.4 kb, corresponding to the full-length receptor and soluble receptor, respectively. Full length VEGFR-1 is an approximately 180 kDa glycoprotein featuring seven extracellular immunoglobulin (Ig)-like domains, a membrane spanning region, and an intracellular tyrosine kinase domain containing a kinase insert sequence. The truncated sVEGFR-1 consists of only first six extracellular Ig-like domains. Ligand binding takes place within the first three N-terminal Ig-like domains while the fourth Ig-like domain is responsible for receptor dimerization, which is a prerequisite for activation through transphosphorylation.

In addition to homodimers, VEGFR-1 can form active heterodimers with VEGFR-2. The soluble form of VEGFR-1 forms inactive heterodimers with VEGFR-2. VEGFR-2 is known as KDR (kinase insert domain receptor) in humans or FLK-1 (fetal liver kinase-1) in mice. Like VEGFR-1, VEGFR-2 also contains seven Ig-like repeats within its extracellular domains and kinase insert domains in its intracellular regions. It is the aim of the invention to block activity of VEGF on these receptors since these receptors play essential roles in pathological angiogenesis. VEGFR-2 binds VEGF-A (VEGF121, VEGF165, VEGF189 and VEGF206 splice variants), VEGF-C and VEGF-D. Full-length cDNA for VEGFR-2 encodes a 1356 amino acid (aa) precursor protein with a 19 aa signal peptide. The mature protein is composed of a 745 aa extracellular domain, a 25 aa transmembrane domain and a 567 aa cytoplasmic domain. In contrast to VEGFR-1, which binds both PIGF and VEGF with high affinity, VEGFR-2 binds VEGF with high affinity but not PIGF. Soluble forms of VEGFR-1 and VEGFR-2 also differ significantly from one another in terms of their abilities to block VEGF-induced cell proliferation and migration. Soluble VEGFR-2 cannot compete with soluble VEGFR-1 for binding with VEGF in human endothelial cells expressing both VEGFR-1 and VEGFR-2. This is because soluble VEGFR-2 can only partially inhibit cell migration, whereas soluble VEGFR-1 can almost completely block VEGF-induced cell proliferation and migration.

1. A method of treating aberrant ocular angiogenesis comprising:
a) obtaining a population of endothelial cells;
b) endowing replicative capacity on the endothelial cells in a manner to resemble VEGF-driven ocular aberrant angiogenesis;
c) exposing the endothelial cells under conditions resembling wet macular degeneration/diabetic nephropathy proliferative microenvironment;
d) treating the endothelial cells with agents capable of increasing immunogenicity of the endothelial cells; and
e) administering the endothelial cells in a manner to stimulate an immune response against the endothelial cells, as well as an immune response capable of recognizing endothelial cells comprising aberrant ocular vascular in conditions such as wet macular degeneration or diabetic retinopathy.

2. The method of claim 1, wherein the endothelial cells are derived from the placenta.

3. The method of claim 2, wherein the placenta is allogeneic to the recipient.

4. The method of claim 2, wherein the endothelial cells are derived from the chorionic portion of the placenta.

5. The method of claim 4, wherein the endothelial cells are derived from the perivascular area of the chorionic portion of the placenta.

6. The method of claim 1, wherein the endothelial cells are generated from a pluripotent stem cell population.

7. The method of claim 6, wherein the pluripotent stem cell population is selected from a group of cells comprising:
a) embryonic stem cells;
b) inducible pluripotent stem cells;
c) somatic cell nuclear transfer generated stem cells; and
d) parthenogenic stem cells.

8. The method of claim 1, wherein the endothelial cells are generated from endothelial precursor cells.

9. The method of claim 8, wherein the endothelial precursor cells are obtained from a population of cells selected from a group comprising of:
a) peripheral blood mononuclear cells;
b) adipose tissue derived stromal vascular fraction;
c) umbilical cord blood;
d) perivascular tissue obtained from the wharton's jelly; and
e) perivascular tissue obtained from the omentum.

10. The method of claim 8, wherein the endothelial precursor cells possess expression of the marker kdr-1.

11. The method of claim 1, wherein the replicative capacity is endowed by culture in a media containing mitogens.

12. The method of claim 11, wherein the mitogens comprise growth factors.

13. The method of claim 11, wherein the mitogen is fetal calf serum.

14. The method of claim 11, wherein the mitogen is human serum.

15. The method of claim 11, wherein the mitogen is high glucose conditions.

16. The method of claim 11, wherein the mitogen is high VEGF.

17. The method of claim 11, wherein the mitogen is selected from a group of mitogens comprising:
a) VEGF;
b) IGF-;
c) FGF-1;
d) FGF-2;
e) TGF-alpha;
f) FGF-5;
g) PDGF;
h) EGF;
i) IL-13;
j) IL-20;
k) NGF;
l) BDNF; and
m) HGF.

18. The method of claim 1, wherein the endothelial cells induced to proliferate resemble ocular hyperproliferative endothelial cells.

19. The method of claim 1, wherein the endothelial cells are treated under conditions of hyperglycemia to endow replication of characteristics of ocular endothelial cells in conditions of diabetic retinopathy.

20. The method of claim 1, wherein the characteristics of the wet macular regeneration/diabetic retinopathy endothelial cells are expression of a marker selected from a group comprising:
a) TEM-1;
b) Robo 1-8;
c) VEGF-r; and
d) endosialin.
